(19) United States
(12) Patent Application Publication
(10) Pub. No.: US 2006/0135933 A1
(43) Pub. Date: Jun. 22, 2006

(54) Stretchable absorbent article featuring a stretchable segmented absorbent

(76) Inventors: Seth M. Newlin, Paris, TX (US); Davis-Dang H. Nhan, Appleton, WI (US)

Correspondence Address: KIMBERLY-CLARK WORLDWIDE, INC., 401 NORTH LAKE STREET, NEENAH, WI, 54956, US

(21) Appl. No.: 11/020,843

(22) Filed: Dec. 21, 2004

(51) Int. Cl. A61F 13/15

(52) U.S. Cl. 604/385.22

(57) ABSTRACT

Disclosed is an absorbent article including a stretchable, liquid impermeable outercover, a stretchable, liquid permeable liner generally superposed with the outercover and an absorbent structure disposed between the liner and the outercover. The absorbent structure includes an absorbent body including at least two segments.

### BACKGROUND OF THE INVENTION

This invention relates generally to absorbent articles such as training pants, diapers, feminine hygiene products, incontinence garments and the like, and more particularly to such articles having a stretchable segmented absorbent.

Absorbent articles such as diapers, training pants, incontinence garments, and the like often include stretchable portions such as leg elastics, waist elastics, elastomeric ears and/or stretchable side panels. These components typically improve the fit of the article upon the wearer, and thus, improve the comfort of the product as well as the ability of the article to contain bodily exudates. Despite the use of these stretchable components, there is a desire to provide absorbent articles with stretchable outercovers and stretchable liners to still further improve the fit, comfort and performance of the absorbent articles.

As can be readily appreciated, absorbent articles typically include an absorbent structure to absorb and retain liquid body exudates, such as urine. Absorbent structures well known in the art can include a matrix of absorbent fiber material such as cellulosic fluff and superabsorbent material; typically, these conventional absorbent structures perform satisfactorily.

Nonetheless, such absorbent structures may not be completely satisfactory when used in connection with an article including a stretchable outercover and/or a stretchable liner. For example, if the absorbent structure is included in an article having a stretchable outercover and/or a stretchable liner, the absorbent structure can have a tendency to shift within the absorbent article, to tear, or to otherwise become permanently distorted, all of which can reduce the intended absorbent characteristics of the absorbent structure and increase the possibility of liquid exudates leaking from the article. Moreover, securing a conventional absorbent structure to the stretchable outercover and/or liner can tend to reduce the stretchability of the substrate to which the absorbent structure is secured, thereby reducing the effectiveness of these stretchable components.

There is need, therefore, to provide an absorbent article including a stretchable outercover, a stretchable liner, and an absorbent structure that is capable of accommodating the stretchability of the stretchable components and/or layers that, in part, make up the article.

### SUMMARY OF THE INVENTION

In one aspect, the present invention is directed to an absorbent article including a stretchable, liquid impermeable outercover, a stretchable, liquid permeable liner generally superposed with the outercover, and an absorbent structure disposed between the liner and the outercover. The absorbent structure includes an absorbent body including at least two absorbent segments and a stretchable, liquid permeable absorbent body wrapsheet substantially surrounding the absorbent body where the absorbent body wrapsheet is attached to the absorbent body.

In another aspect, the present invention is directed to an absorbent article including an elastic, liquid impermeable outercover, an elastic, liquid permeable liner generally superposed with the outercover, and an absorbent structure disposed between the liner and the outercover. The absorbent structure includes an absorbent body including at least two absorbent segments and a stretchable, liquid permeable absorbent body wrapsheet substantially surrounding the absorbent body where the absorbent body wrapsheet is attached to the absorbent body.

In yet another aspect, the present invention is directed to an absorbent article defining a lateral direction and a longitudinal direction. The absorbent article includes an elastic, liquid impermeable outercover, an elastic, liquid permeable liner generally superposed with the outercover, and an absorbent structure disposed between the liner and the outercover. The absorbent structure includes an absorbent body including an absorbent segment matrix of at least 2 absorbent segments in the lateral direction and at least 5 absorbent segments in the longitudinal direction. The absorbent structure also includes a stretchable, liquid permeable absorbent body wrapsheet substantially surrounding the absorbent body wherein the absorbent body wrapsheet is attached to the absorbent body.

In still yet another aspect the present invention is directed to an absorbent article defining a lateral direction and a longitudinal direction. The absorbent article includes an elastic, liquid impermeable outercover, an elastic, liquid permeable liner generally superposed with the outercover, and an absorbent structure disposed between the liner and the outercover. The absorbent structure includes an absorbent body including an absorbent segment matrix of at least 2 absorbent segments in the lateral direction and at least 5 absorbent segments in the longitudinal direction. The absorbent structure also includes a stretchable, liquid permeable absorbent body wrapsheet substantially surrounding the absorbent body wherein the absorbent body wrapsheet is attached to the absorbent body.

The above-mentioned and other aspects of the present invention will become more apparent, and the invention itself will be better understood by reference to the drawings and the following description of the drawings.

### BRIEF DESCRIPTION OF THE DRAWINGS

FIG. 1 representatively illustrates a side view of a pair of training pants with a mechanical fastening system of the pants shown fastened on one side of the training pants and unfastened on the other side of the training pants;

FIG. 2 representatively illustrates a plan view of the training pants of FIG. 1 in an unfastened, stretched and laid flat condition, and showing the surface of the training pants that faces away from the wearer;

FIG. 3 representatively illustrates a plan view similar to FIG. 2, but showing the surface of the training pants that faces the wearer when worn, and with portions cut away to show underlying features;

FIG. 4 representatively illustrates a plan view similar to FIG. 3 with portions cut away to show features of the absorbent structure;

FIG. 5 representatively illustrates a plan view similar to FIG. 4 of another aspect of the present invention;

FIGS. 6 and 7 representatively illustrate section views of various aspects of the absorbent structures of the present invention in a relaxed state; and

FIGS. 8 and 9 representatively illustrate section views of various aspects of the absorbent structures of the present invention in an extended state.

Corresponding reference characters indicate corresponding parts throughout the drawings.

### DEFINITIONS

Within the context of this specification, each term or phrase below includes the following meaning or meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” or “disposed toward” and variations thereof are intended to mean that one element can be integral with another element, or that one element can be a separate structure bonded to or placed with or placed near another element.

“Elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 25 percent (to 125 percent) of its relaxed length and will recover, upon release of the applied force, at least 40 percent of its elongation. Desirably an elastic material or composite be capable of being elongated by at least 100 percent (to 200 percent), more desirably by at least 150 percent (to 250 percent), of its relaxed length and recover, upon release of an applied force, at least 50 percent of its elongation.

“Extensible” refers to a material or composite which is capable of extension or deformation without breaking, but does not substantially recover its original size and shape after removal of a force causing the extension or deformation. Suitably, an extensible material or composite can be elongated by at least 25 percent (to 125 percent) of its relaxed length. Desirably an extensible material or composite be capable of being elongated by at least 100 percent (to 200 percent), more desirably by at least 150 percent (to 250 percent), of its relaxed length.

“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Stretchable” means that a material can be stretched, without breaking, by at least 25 percent (to 125 percent of its initial (unstretched) length) in at least one direction, suitably by at least 100 percent (to 200 percent of its initial length), desirably by at least 150 percent (to at least 250 percent of its initial length) and may or may not recover properties upon release of an applied force. Elastic materials and extensible materials are each stretchable materials.

“superabsorbent material” refers to a water-swellable, water-insoluble organic or inorganic material capable, under the most favorable conditions, of absorbing at least about ten times its weight and, more desirably, at least about thirty times its weight in an aqueous solution containing about 0.9 weight percent sodium chloride.

These terms may be defined with additional language in the remaining portions of the specification.

### DETAILED DESCRIPTION

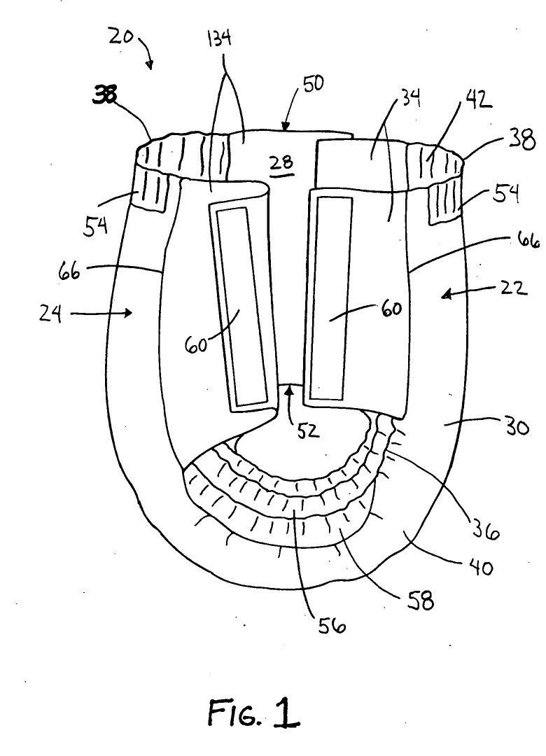

Referring now to the drawings and in particular to FIG. 1, an absorbent article of the present invention is representatively illustrated in the form of children's toilet training pants and is indicated in its entirety by the reference numeral 20. The absorbent article 20 may or may not be disposable, which refers to articles that are intended to be discarded after a limited period of use instead of being laundered or otherwise conditioned for reuse. It is understood that the present invention is suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing training pants such as the pants 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al., the disclosures of which are incorporated herein by reference to the extent that they are consistent (i.e., not in conflict) herewith.

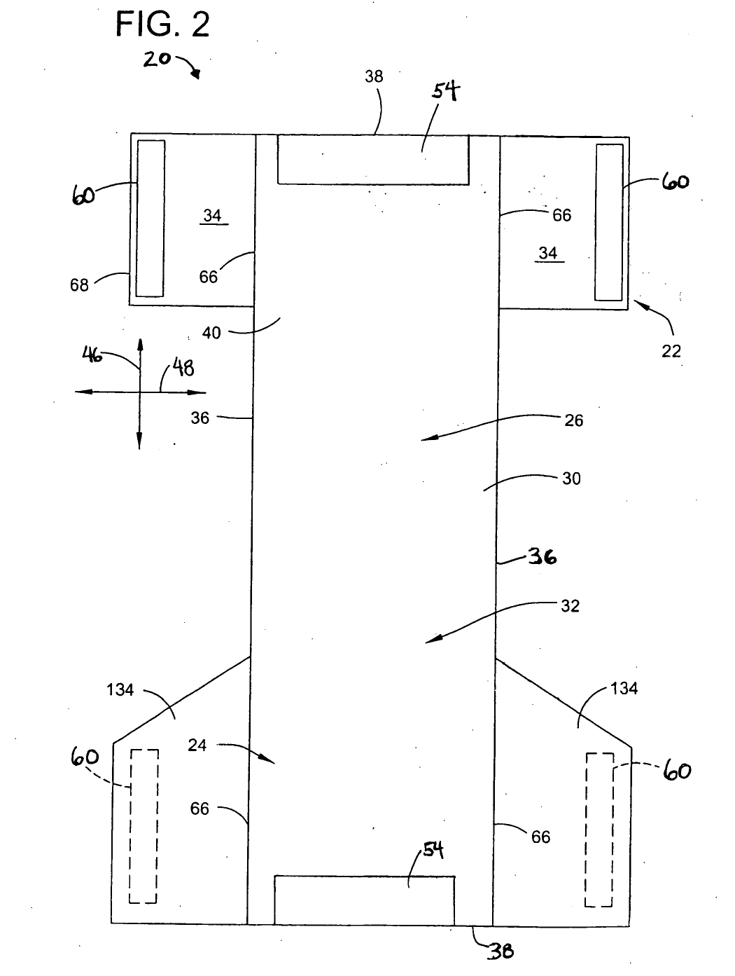

The pair of training pants 20 is illustrated in FIG. 1 in a partially fastened condition. The pants 20 define a longitudinal direction 46 and a lateral direction 48 perpendicular to the longitudinal direction as shown in FIGS. 2-5. The pants 20 further define a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The front and back waist regions 22, 24 includes those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The pants 20 also define an inner surface 28 adapted in use to be disposed toward the wearer, and an outer surface 30 opposite the inner surface. With additional reference to FIGS. 2-5, the pair of training pants 20 has a pair of laterally opposite side edges 36 and a pair of longitudinally opposite waist edges 38 (broadly, longitudinal ends).

The illustrated pants 20 can include an absorbent assembly, generally indicated at 32. For example, in the aspect of FIGS. 1-5, the training pants 20 include a generally rectangular central absorbent assembly 32 and side panels 34, 134 formed separately from and secured to the central absorbent assembly. The side panels 34, 134 are bonded along seams 66 to the absorbent assembly 32 in the respective front and back waist regions 22 and 24 of the pants 20. More particularly, the front side panels 34 can be permanently bonded to and extend laterally outward from the absorbent assembly 32 at the front waist region 22, and the back side panels 134 can be permanently bonded to and extend laterally from the absorbent assembly 32 at the back waist region 24. The side panels 34 and 134 may be bonded to the absorbent assembly 32 using attachment means known to those skilled in the art such as adhesive, thermal or ultrasonic bonding.

The front and back side panels 34 and 134, upon wearing of the pants 20, thus include the portions of the training pants 20 that are positioned on the hips of the wearer. The front and back side panels 34 and 134 can be permanently bonded together to form the three-dimensional configuration of the pants 20, or be releasably connected with one another such as by a fastening system 60 of the illustrated aspects.

Suitable elastic materials, as well as one process of incorporating elastic side panels into training pants, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; the disclosures of which are incorporated herein by reference to the extent they are consistent (i.e., not in conflict) herewith. In particular aspects, the elastic material may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; the disclosures of which are incorporated herein by reference to the extent they are consistent (i.e., not in conflict) herewith. As is known in the art, the side panels 34, 134 may include elastic material or stretchable but inelastic materials.

The absorbent assembly 32 is illustrated in FIGS. 1-5 as having a rectangular shape. However, it is contemplated that the absorbent assembly 32 may have other shapes (e.g., hourglass, T-shaped, I-shaped, and the like) without departing from the scope of this invention. It is also understood that the side panels 34, 134 may instead be formed integrally with the absorbent assembly 32 without departing from the scope of this invention. In such a configuration, the side panels 34 and 134 and the absorbent assembly would include at least some common materials, such as the bodyside liner 42, outercover 40, other materials and/or combinations thereof.

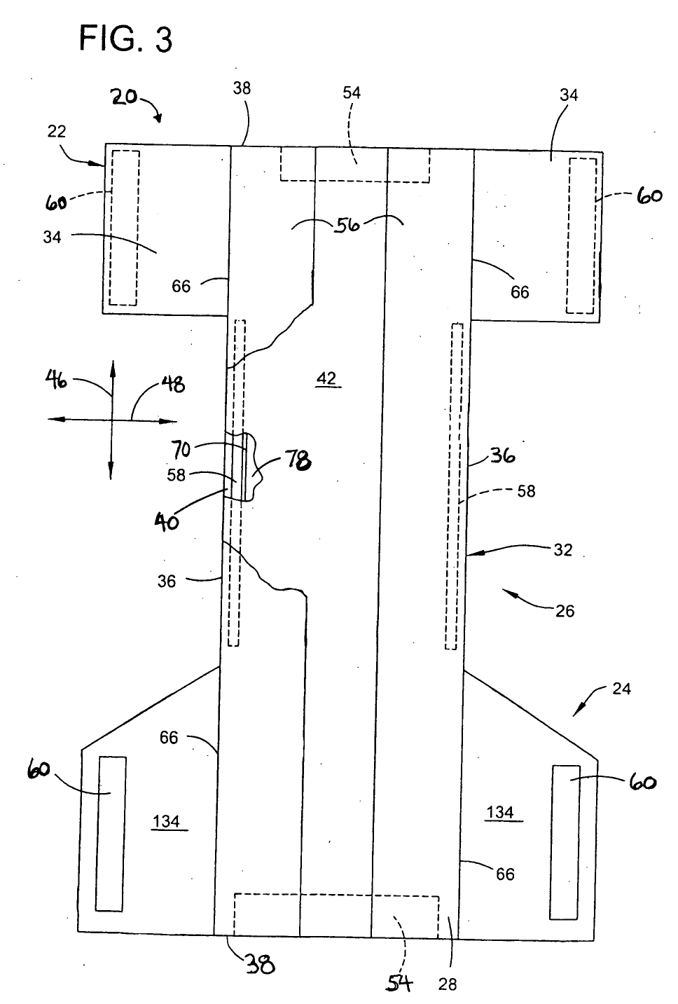

The absorbent assembly 32 includes an outercover 40 and a bodyside liner 42 (FIGS. 1 and 3) in a superposed relation therewith. The absorbent assembly 32 also includes an absorbent structure 70 (FIGS. 3-5) disposed between the outercover 40 and the bodyside liner 42 for absorbing liquid body exudates. As will be described in greater detail below, the absorbent structure 70 can be stretchable and therefore particularly adapted for use in pants 20 having a stretchable outercover 40 and a stretchable liner 42. The liner 42 can be suitably joined to the outercover 40 along at least a portion of the longitudinal ends of the pants 20. The bodyside liner 42 and the outercover 40 can, for example, be attached to each other by adhesive, ultrasonic bonding, thermal bonding or by other suitable attachment techniques known in the art. Moreover, at least a portion of the absorbent structure 70 can optionally be attached to the bodyside liner 42 and/or the outercover 40 utilizing the methods described above.

As mentioned above, the front and back side panels 34 and 134 can be releasably connected with one another such as by the fastening system 60 of the illustrated aspect. With the training pants 20 in the fastened position as partially illustrated in FIG. 1, the front and back waist regions are connected together to define the three-dimensional pants configuration having a waist opening 50 and a pair of leg openings 52. The waist edges 38 of the training pants 20 are configured to encircle the waist of the wearer to define the waist opening 50 (FIG. 1) of the pants.

The fastening system 60 may include any refastenable fasteners suitable for absorbent articles, such as adhesive fasteners, cohesive fasteners, mechanical fasteners, or the like. In one aspect of the invention, the fastening system includes mechanical fastening elements for improved performance. Suitable mechanical fastening elements can be provided by interlocking geometric-shaped materials, such as hooks, loops, bulbs, mushrooms, arrowheads, balls on stems, male and female mating components, buckles, snaps, or the like. For example, fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

The pants 20 may further include a pair of containment flaps 56 for inhibiting the lateral flow of body exudates. As representatively illustrated in FIG. 3, the containment flaps 56 can be operatively attached to the pants 20 in any suitable manner as is well known in the art. In particular, suitable constructions and arrangements for the containment flaps 56 are generally well known to those skilled in the art and are described in U.S. Pat. No. 4,704,116 issued Nov. 3, 1987 to Enloe, which is incorporated herein by reference to the extent that it is consistent (i.e., not in conflict) herewith.

To further enhance containment and/or absorption of body exudates, the training pants 20 may include waist elastic members 54 in the front and/or back waist regions 22 and 24 of the pants 20. Likewise, the pants 20 may include leg elastic members 58, as are known to those skilled in the art. The waist elastic members 54 and the leg elastic members 58 can be formed of any suitable elastic material that is well known to those skilled in the art. For example, suitable elastic materials include sheets, strands or ribbons of natural rubber, synthetic rubber, or thermoplastic elastomeric polymers. In one aspect of the invention, the waist elastics and/or the leg elastics may include a plurality of dry-spun coalesced multi-filament spandex elastomeric threads sold under the trade name LYCRA and available from Invista of Wilmington, Del., U.S.A.

The outercover 40 can suitably include a material that is substantially liquid impermeable. The outercover 40 may be provided by a single layer of liquid impermeable material, or more suitably include a multi-layered laminate structure in which at least one of the layers is liquid impermeable. In particular aspects, the outer layer may suitably provide a relatively cloth-like texture to the wearer. Alternatively, the outercover 40 may include a woven or non-woven fibrous web layer that has been totally or partially constructed or treated to impart the desired levels of liquid impermeability to selected regions that are adjacent or proximate the absorbent structure. Suitably, the outercover 40 is stretchable, and even more suitably the outercover is elastic.

As an example, the outercover 40 may include cast or blown films, foams, or meltblown fabrics composed of polyethylene, polypropylene, or polyolefin copolymers, as well as combinations thereof. The elastomeric materials may include PEBAX® elastomer (available from AtoChem located in Philadelphia, Pa.), HYTREL® elastomeric polyester (available from E. I. DuPont de Nemours located in Wilmington, Del.), KRATON® elastomer (available from Kraton Polymers located in Houston, Tex.), or strands of LYCRA® elastomer (available from Invista located in Wilmington, Del.), or the like, as well as combinations thereof. The outercover 40 may include materials that have elastomeric properties imparted by a mechanical process, a printing process, a heating process, and/or a chemical treatment. For example, such materials may be apertured, creped, neck-stretched, heat activated, embossed, micro-strained, or a combination thereof.

In particular aspects of the invention, the outercover 40 may include a 0.4 ounces per square yard (osy) (13.6 grams per square meter (gsm)) basis weight layer of G2760 KRATON elastomer strands adhesively laminated with a 0.3 gsm layer of adhesive between two facings. Each facing can be composed of a thermal point bonded bicomponent spunbond non-woven fibrous web having a 0.7 osy (23.7 gsm) basis weight. The adhesive is similar to an adhesive which is supplied by Bostik-Findley Adhesive and designated as H2525A, and the elastomer strands are placed and distributed to provide approximately 12 strands of KRATON elastomer per inch (2.54 cm) of lateral width of the outercover 40.

The pants 20 of the present invention can alternatively include a biaxially stretchable outercover 40. For example, such an outercover material can include a 0.3 osy polypropylene spunbond that is necked 60 percent in the lateral direction 40 and creped 60 percent in the longitudinal direction 46, laminated with 3 grams per square meter (gsm) Bostik-Findley H2525A styrene-isoprene-styrene based adhesive to 8 gsm PEBAX 2533 film with 20 percent TiO₂ concentrate.

Additional examples of a stretchable outercover 40 are provided in U.S. Pat. No. 5,883,028, issued to Morman et al.; U.S. Pat. No. 5,116,662, issued to Morman and U.S. Pat. No. 5,114,781, issued to Morman; the disclosures of which are incorporated herein by reference to the extent that they are consistent (i.e., not in conflict) herewith.

Suitably, the outercover 40 of the present invention can be stretched, laterally and/or longitudinally, by at least 30 percent (to at least 130 percent of an initial (unstretched) width and/or length of the outercover 40). More suitably, the outercover 40 can be stretched laterally and/or longitudinally, by at least 50 percent (to at least 150 percent of the unstretched width or length of the outercover 40). Even more suitably, the outercover 40 can be stretched, laterally and/or longitudinally, by at least 100 percent (to at least 200 percent of the unstretched width or length of the outercover 40). Tension force in the outercover 40 at 50 percent extension is suitably between 50 and 1000 grams, more suitably between 100 and 600 grams, as measured on a 3 inch (7.62 cm) wide piece of the outercover material.

The bodyside liner 42 of the present invention is suitably compliant, soft-feeling, and non-irritating to the wearer's skin. The bodyside liner 42 is also sufficiently liquid permeable to permit liquid body exudates to readily penetrate through its thickness to the absorbent structure 70.

In a particular aspect, the bodyside liner 42 is stretchable, and more suitably elastic. For example, the stretchable bodyside liner 42 can include elastic strands, cast or blown elastic films, non-woven elastic webs, meltblown or spunbond elastomeric fibrous webs, as well as combinations thereof. Examples of suitable elastomers include those described above as suitable for use in an elastomeric outercover 40.

For instance, the liner 42 can be a non-woven, spunbond polypropylene fabric composed of about 2 to 3 denier fibers formed into a web having a basis weight of about 12 gsm which is necked approximately 60 percent. Strands of about 9 gsm KRATON G2760 elastomer material placed eight strands per inch (2.54 cm) can be adhered to the necked spunbond material to impart elasticity to the spunbond fabric. The fabric can be surface treated with an operative amount of surfactant, such as about 0.6 percent AHCOVEL Base N62 surfactant, available from ICI Americas, a business having offices in Wilmington, Del., U.S.A. Other suitable materials may be extensible biaxially stretchable materials, such as a neck stretched/creped spunbond. Stretchable bodyside liners 42 are also described in U.S. Pat. No. 6,552,245, issued Apr. 22, 2003, to Roessler et al., the disclosure of which is incorporated herein by reference to the extent that it is consistent (i.e., not in conflict) herewith.

In particular aspects, the liner 42 of the present invention can suitably be stretched, laterally and/or longitudinally, by at least 30 percent (to at least 130 percent of an initial (unstretched) width and/or length of the liner 42). More suitably, the liner 42 can be stretched laterally and/or longitudinally, by at least 50 percent (to at least 150 percent of the unstretched width or length of the liner 42). Even more suitably, the liner 42 can be stretched, laterally and/or longitudinally, by at least 100 percent (to at least 200 percent of the unstretched width or length of the liner 42). Tension force in the liner 42 at 50 percent extension is suitably between 50 and 1000 grams, more suitably between 100 and 600 grams, as measured on a 3 inch (7.62 cm) wide piece of the liner material.

The absorbent structure 70 of the various aspects of the present invention can include an absorbent body 72 and a stretchable absorbent body wrapsheet 78. Further, the absorbent body 72 can include a plurality (that is, at least two) absorbent segments and boundary regions 76 located between adjacent absorbent segments. The boundary regions 76 are suitably constructed to allow the absorbent segments 74 to separate from each other in use. Moreover, the absorbent structure 70 can be suitably stretchable, and in particular aspects may be elastic in at least one of the longitudinal and lateral directions 46 and 48, and optionally both the longitudinal and lateral directions 46 and 48.

The absorbent structure 70 of the present invention may be a variety of shapes as are known in the art. For example, as representatively illustrated in FIGS. 3-5, the absorbent structure 70 is generally rectangular in shape. Alternatively, the absorbent structure 70 may be I-shaped, hourglass shaped, or the like.

The absorbent structure 70 is suitably conformable, non-irritating to a wearer's skin, and capable of absorbing and retaining liquids and certain body wastes. As such, the absorbent body 72 of the absorbent structure 70 may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In addition, superabsorbent material may be suitably present in the absorbent body 72 in an amount of from about 0 to about 99 weight percent based on total weight of the absorbent body 72.

The absorbent materials may be formed into an absorbent web structure by employing various conventional methods and techniques known in the art. For example, the absorbent body 72 may be formed by a dry-forming technique, an air forming technique, a wet-forming technique, a foam-forming technique, or the like, as well as combinations thereof. Methods and apparatus for carrying out such techniques are well known in the art. The absorbent body 72 may alternatively include a coform material such as the material disclosed in U.S. Pat. No. 4,100,324 to Anderson, et al.; U.S. Pat. No. 5,284,703 to Everhart, et al.; and U.S. Pat. No. 5,350,624 to Georger, et al.; the disclosures of which are incorporated herein by reference to the extent that they are consistent (i.e., not in conflict) herewith.

Alternatively, the absorbent body 72 may be stretchable. For example, the absorbent body 72 may include materials disclosed in U.S. Pat. No. 5,964,743, issued Oct. 12, 1999, to Abuto et al.; U.S. Pat. No. 6,231,557, issued May 15, 2001, to Krautkramer et al.; U.S. Pat. No. 6,362,389, issued Mar. 26, 2002, to McDowall et al.; and international patent application WO 03/051254, published Jun. 26, 2003 in the name of Uitenbroek et al.; the disclosures of which are incorporated by reference herein to the extent that they are consistent (i.e., not in conflict) herewith.

As mentioned above, the absorbent body 72 includes a plurality of absorbent segments 74 (that is, at least two, and in the illustrated embodiments more than two). Desirably, the absorbent body includes at least four absorbent segments 74. At least one boundary region 76 is located between and thus separates adjacent absorbent segments 74 of the absorbent body 72. In the illustrated aspects, the absorbent body 72 includes a plurality of boundary regions 76. Thus, the absorbent segments 74 are positioned generally adjacent each other in a plane defined by the lateral direction 48 and the longitudinal direction 46 to provide the absorbent body 72.

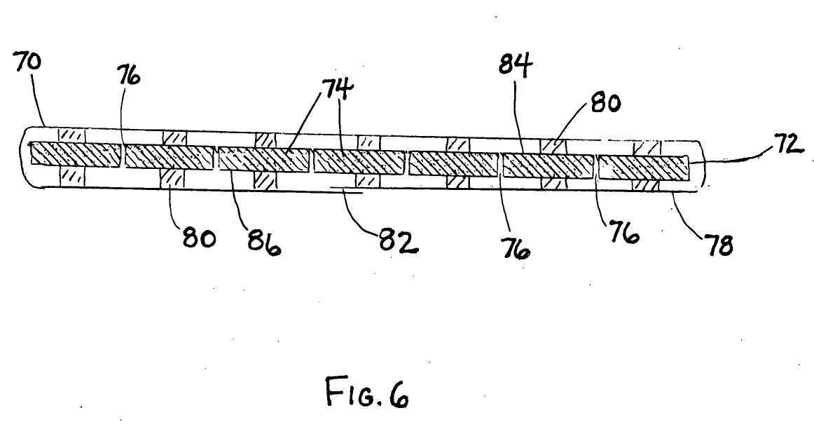

Adjacent absorbent segments 74 can be discrete (e.g., detached, or non-interconnected) as representatively illustrated in FIG. 6 and thus be spaced from each other in order to provide the boundary region 76. In a particular aspect, the absorbent segments 74 can be spaced in at least one of the lateral direction 48 and the longitudinal direction 46 to provide the boundary regions 76.

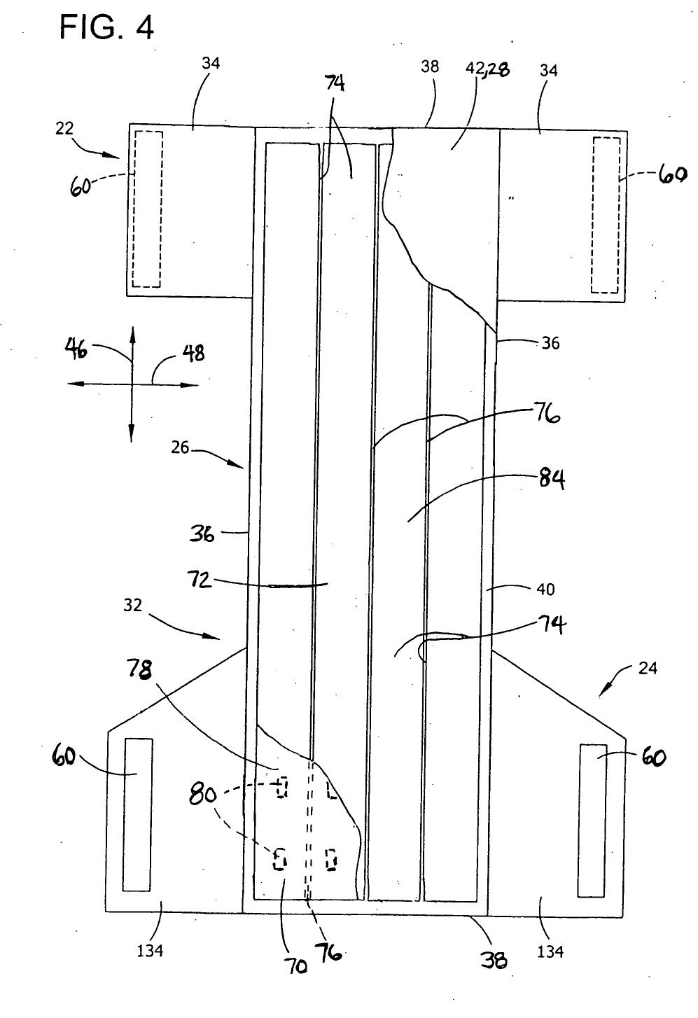

As such, the absorbent segments 74 can be separate discrete elements spaced from each other whereby the boundary regions 76 include the spacing between adjacent absorbent segments 74. It is also contemplated that adjacent yet discrete absorbent segments 74 can be in an abutting relationship. Thus, when the absorbent structure 70 is in a relaxed or otherwise non-stretched condition as shown in FIGS. 4-7, the boundary regions 76, that is, the spacing between adjacent absorbent segments 74, are suitably less than about 5 millimeters (mm) and are more suitably in the range of 0 to about 3 mm. However, it is understood that the spacing between adjacent absorbent segments 74 of the outer absorbent body 72 may be greater than about 5 mm without departing from the scope of the invention.

An absorbent body 72 may be constructed into discrete absorbent segments 74 by cutting a formed absorbent web into the discrete absorbent segments 74. For example, the absorbent body 72 may be a conventional air-formed absorbent. Alternatively, each absorbent segment 74 may be formed separately and arranged relative to each other in the desired arrangement.

Alternatively, the boundary regions 76 between the absorbent segments 74 may be configured to separably join adjacent absorbent segments 74 of absorbent body 72. In such an arrangement, the boundary regions 76 are suitably constructed to permit movement of adjacent absorbent segments 74 relative to each other upon the application of a force to the absorbent structure 70 such as by flexing at the boundary regions 76 or by breaking the joinder of the segments 74 at the boundary regions 76.

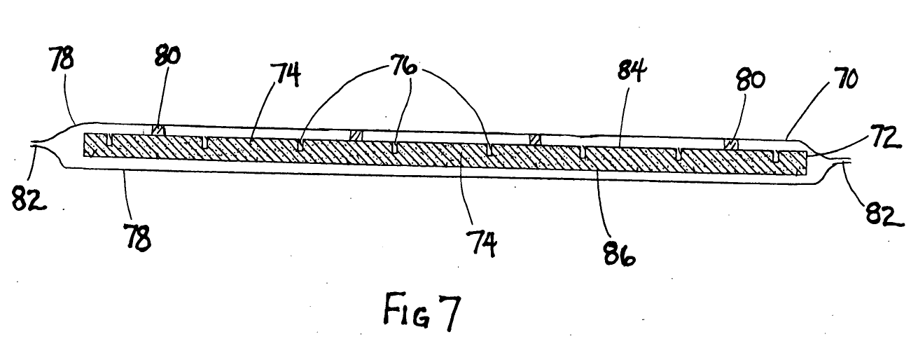

For example, as representatively illustrated in FIG. 7, the absorbent segments may be joined and yet be readily separable within the boundary regions 76 upon the application of an extension force to the absorbent structure 70. As such, upon stretching of the outercover 40 and/or the liner 42, the absorbent segments move with the pants outercover 40 and/or the liner 42 to further separate from each other generally at the boundary regions 76. In one aspect, the outer absorbent body 72 can have a density at at least one boundary region 76 that is substantially less than the density of the adjacent absorbent segments 74. In another embodiment, the absorbent body may have a basis weight at at least one boundary region 76 that is substantially less than the basis weight of the adjacent absorbent segment 74.

One suitable method of forming such an absorbent body as described above is to insert an additional wire mesh screen (not shown) over the forming surface of a conventional air-forming device (not shown). As fibers and superabsorbent material are collected on the forming surface to form the absorbent structure, a lesser amount of material is collected on the forming surface at the wires of the additional wire screen. The formed absorbent body 72 may then appear as illustrated in FIG. 7 having absorbent segments 74 interconnected by boundary regions 76 (e.g., where the wires of the additional wire screen were located) whereby the boundary regions 76 have a lower basis weight than the absorbent segments.

The absorbent body 72 may be further processed, such as by passing the absorbent structure through a nip defined by opposed rolls in order to compress the absorbent body 72. Following compression in this manner, the boundary regions 76 of the absorbent body 72 have a lower density than the absorbent segments 74 of the absorbent body 72.

In yet another alternative, the absorbent body 72 may include boundary regions 76 between the absorbent segments 74 thereof that are a combination of both spacing between adjacent absorbent segments 74, and separable joinder between adjacent absorbent segments 74. Accordingly, the boundary regions 76 between adjacent absorbent segments are suitably constructed such that upon the application of an elongating force to the outercover 40 and/or the liner 42, the absorbent segments 74 separate from each other generally at the boundary regions 76 thereby allowing the absorbent structure 70 to generally move with the outercover 40 and/or the liner 42.

The absorbent segments 74 may be provided in a variety of shapes and configurations in order to provide the absorbent body 72 with the desired shape and the absorbent structure 70 with the desired functionality. That is, the absorbent segments 74 may be square, triangular, diamond shaped, or other suitable shapes and combinations thereof. In addition, it is understood that the absorbent segments 74 may have different lengths and/or widths and or sizes relative to each other, or may generally be the same size relative to each other.

For example, as representatively illustrated in FIG. 4, the absorbent segments 72 are generally elongate and rectangular. As such, the laterally opposite side edges of the absorbent segments 72 are disposed in generally edge-facing-edge relationship with a corresponding side edge of at least one adjacent absorbent segment 72.

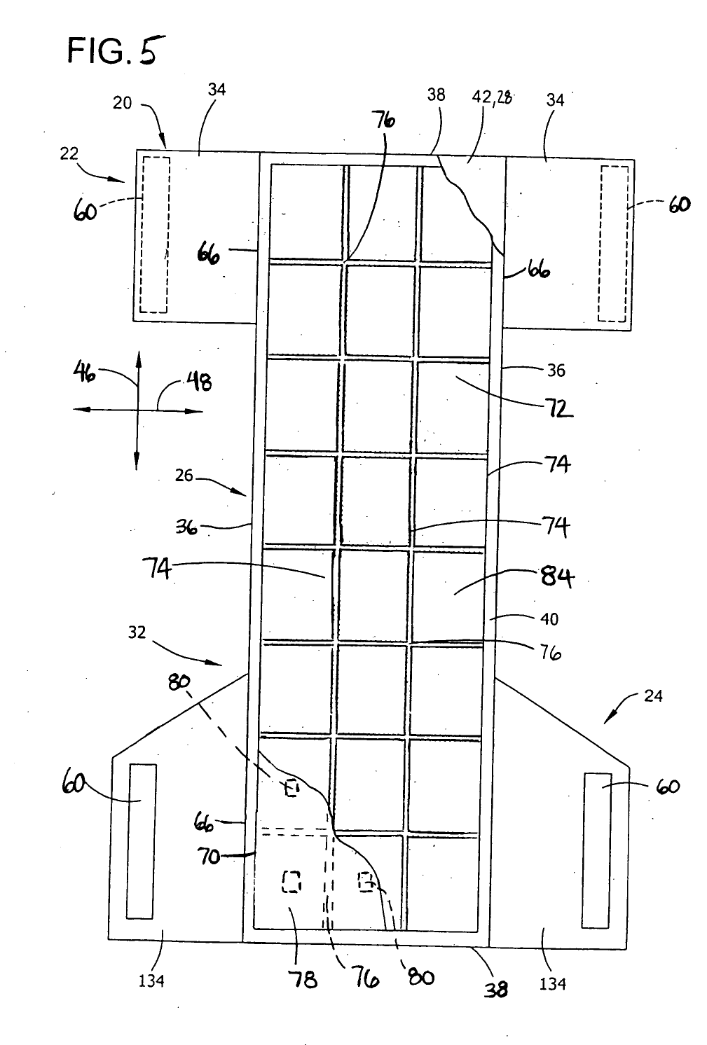

Alternatively, the absorbent body 72 can include a matrix of absorbent segments 74. For example, as representatively illustrated in FIG. 5, the absorbent segments 74 can be generally rectangular and arranged so that the boundary regions 76 between adjacent absorbent segments 74 extend in both the lateral direction 48 and the longitudinal direction 46 to provide a matrix of absorbent segments 74. In particular, the absorbent segments 74 may each have a length in the range of about 1 cm to about 5 cm, and more suitably a length of about 2.5 cm. Likewise, the absorbent segments 74 may have a width in the range of about 1 cm to about 5 cm.

In a particular aspect, the matrix of absorbent segments 74 can be provided by a grid of at least 2 absorbent segments 74 in the lateral direction 48, and at least 5 absorbent segments 74 in the longitudinal direction 46. For instance, as representatively illustrated in FIG. 5, the matrix may be provided by a grid of 3 absorbent segments 74 in the lateral direction 48, and 8 absorbent segments 74 in the longitudinal direction 46. In addition, it is contemplated that other grid configurations may be employed while remaining within the scope of the present invention.

For instance, a matrix of absorbent segments 74 can be arranged to provide the majority of segments 74 in the waist regions 22 and 24, while having fewer segments or only one segment in the crotch region 26 of the pants 20. In such an arrangement, the stretch capabilities are maximized in the waist regions 22 and 24 where such capabilities are particularly suitable.

The absorbent segments 74 of the absorbent body 72 can all have generally the same basis weight, density and thickness. However, it is understood that some or all of the absorbent segments 74 may have different basis weights, densities and/or thicknesses relative to each other. It is also contemplated that the concentration of superabsorbent material may be non-uniform among some or all of the absorbent segments 74. For example, absorbent segments 90 having a higher concentration of superabsorbent material may be placed in a target region such as the crotch region 26 and absorbent segments 74 having a lower concentration of superabsorbent material may be placed toward the front and back waist regions 22, 24. It is also contemplated that the basis weight, density, thickness and/or superabsorbent material concentration within one or more of the absorbent segments 74 may be non-uniform across the width and/or along the length of the absorbent segment itself.

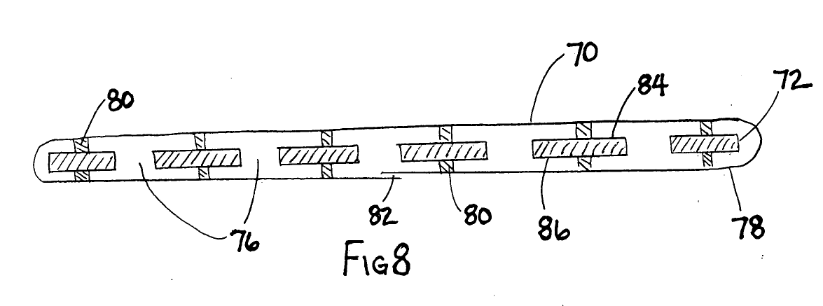

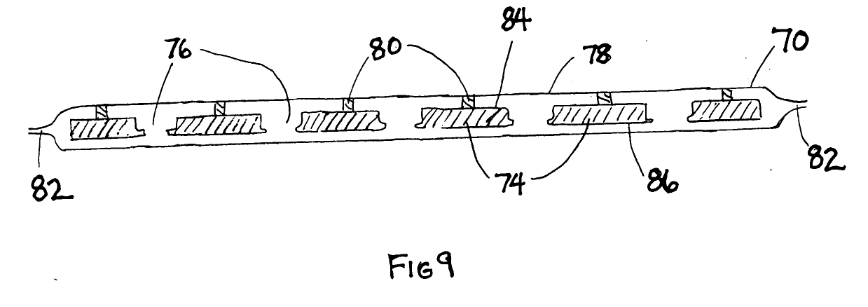

As mentioned above, the absorbent structure 70 of the present invention also includes a substantially liquid permeable, stretchable absorbent body wrapsheet 78. The absorbent body wrapsheet 78 wraps the absorbent body 72 to help maintain the integrity of the absorbent structure 70 and improve the containment of the absorbent material of the absorbent body 72. The wrapsheet 78 can be provided in a number of different configurations that may be contemplated by one of skill in the art. For example, the wrapsheet 78 can be provided by a single piece of material folded about the absorbent body 72. In particular and as representatively illustrated in FIGS. 6 and 8, the wrapsheet 78 can optionally be arranged in a C-fold around the absorbent body 72. In such a configuration, the wrapsheet may optionally be bonded at the wrapsheet seam 82 using adhesives, ultrasonic bonding, pressure bonding, and the like or combinations thereof. Alternatively, as representatively illustrated in FIGS. 7 and 9, the wrapsheet 78 can be provided by multiple pieces of material (e.g. two or more layers) that generally sandwich the absorbent body 72. Optionally, the wrapsheet 78 can be bonded along at least a portion of the wrapsheet seams 82 that, in the instant configuration, are generally located at the perimeter of the absorbent body 72.

Further, the term “wrap” or “wrapping” should not be read to mean necessarily completely wrapping or enveloping only. For example, the absorbent body 72 may define an absorbent body inner surface 84, which is disposed toward the liner 42, and an absorbent body outer surface 86, which is disposed toward the outercover 40, and the absorbent body wrapsheet 78 may cover one of the surfaces 84 and 86, and suitably at least the absorbent body inner surface 84.

Alternatively, as depicted in the illustrated embodiments, the absorbent body wrapsheet 78 may substantially surround the absorbent body 72 and as such cover the absorbent body inner surface 84 and the absorbent body outer surface 86. In such a configuration, the longitudinal end edges of the absorbent body 72 can optionally be left exposed.

Suitable materials for use as a stretchable absorbent body wrapsheet 78 include porous woven materials, porous nonwoven materials (e.g., spunbond and meltblown webs), and apertured films. Further, the wrapsheet 78 may be treated with a surfactant as are known in the art to increase the wettability of the material.

For example, the wrapsheet 78 may be extensible in at least one of the longitudinal and lateral directions 46 and 48. In such a configuration, the wrapsheet 78 can include a necked spunbond material that is extensible in the lateral direction 48 or a necked, creped spunbond material that is extensible in both the longitudinal and lateral direction 46 and 48.

Alternatively, the stretchable absorbent body wrapsheet 78 can be elastic. In particular, the wrapsheet 78 can be elastically stretchable in at least one of the longitudinal and lateral directions 46 and 48. Alternatively, the wrapsheet 78 can be biaxially stretchable and be elastically elongatable in both the longitudinal and lateral directions 46 and 48.

An example of an elastic biaxially stretchable wrapsheet 78 is a bicomponent (sheath/core, with 20 percent by weight polyethylene and 80 percent by weight KRATON elastomer) spunbond web having a basis weight of about 0.8 ounces per square yard (osy) (about 27 grams per square meter, or gsm) and treated with 0.1 percent by weight add on level of a mixture of surfactants (e.g., a 3 to 1 ratio of AHCOVEL surfactant and GLUCOPON surfactant). An Alternative elastomer that may be used in the bicomponent spunbond web include AFFINITY elastomeric polyethylene from Dow Chemical of Midland, Mich., U.S.A.

As mentioned above, the absorbent body 74 can be suitably attached to the absorbent body wrapsheet 78. In particular, at least a portion of the absorbent segments 74 may be attached to the absorbent body wrapsheet 78, such as by adhesive, by thermal or ultrasonic bonding or by other suitable attachment technique, at discrete attachment regions 80 (FIGS. 6-9). Suitably, the absorbent body wrapsheet 78 is directly attached to the absorbent segments 74.

The absorbent body wrapsheet 78 may be attached to substantially all of the absorbent segments 74 of the absorbent body 72, or alternatively, may only be attached to a portion of the absorbent segments 74. For example, at least 10 percent of the absorbent segments 74 can be attached to the absorbent body wrapsheet 78 at discrete attachment regions 80 to advantageously provide the desired absorbent structure integrity in use. Alternatively, at least 20 percent of the absorbent segments 74 can be attached to the absorbent body wrapsheet 78 at attachment regions 80. In another alternative, at least 50 percent of the absorbent segments 74 can be attached to the absorbent body wrapsheet 78 at attachment regions 80.

Further, the discrete attachment regions 80 can be suitably sized (e.g., in length and/or width) smaller than the absorbent segments 74. That is, the attachment regions 80 may each define an attachment region area that does not extend to the longitudinal ends and lateral edges of the absorbent segment 74 to which it is attached, and as such, is less than the absorbent segment area of the absorbent segment to which it is attached. Suitably, the attachment region area can be less than 50 percent of the absorbent segment area. More suitably, the attachment region area can be less than 20 percent of the absorbent segment area. Still more suitably, the attachment region area can be less than 10 percent of the absorbent segment area. Desirably, at least some of the absorbent segments 74 can be attached to the wrapsheet 76 in at least the waist regions 22 and 24.

In such a configuration, the absorbent body wrapsheet 78 may be attached to the absorbent body 72 on at least the absorbent body inner surface 84. Alternatively, the absorbent body wrapsheet 78 may be attached to both the absorbent body inner surface 84 and the absorbent body outer surface 86.

By attaching at least some of the absorbent segments 74 to the stretchable absorbent body wrapsheet 78, the absorbent segments 74 of the absorbent body 72 can move with the wrapsheet 78 when the stretchable outercover and/or liner are elongated by separating at the boundary regions 76. That is, as representatively illustrated in FIGS. 8 and 9, when the wrapsheet 78 is elongated the absorbent segments 74 separate from each other at the boundary regions 76 upon stretching of the pants 20. As such, the absorbent structure 70 may be elongatable in the longitudinal direction and/or the lateral direction 46 and 48. For example, in the aspect illustrated in FIG. 4, the absorbent structure 70 may be elongated in the lateral direction 48. Alternatively, the segments 74 may be oriented in the lateral direction to provide an absorbent structure that is capable of elongating in the longitudinal direction 46.

In yet another alternative, the absorbent structure may be configured to be biaxially stretchable. For example, in the aspect illustrated in FIG. 5, the matrix of absorbent segments 74 attached to a biaxially stretchable wrapsheet 78 provide an absorbent structure 70 that is capable of elongating in the longitudinal direction and the lateral direction 46 and 48.

As can be readily appreciated, the absorbent segments 74 may be arranged in a variety of configurations to provide an absorbent structure 70 that has particular elongation characteristics. Further absorbent segment configurations are described in U.S. patent application Ser. No. 10/698612 filed Oct. 31, 2003 in the name of Kuen, et al., the disclosure of which is incorporated by reference herein to the extent that it is consistent (i.e., not in conflict) herewith.

Moreover, when the stretchable absorbent body wrapsheet 78 is elastomeric, the absorbent structure 70 can be elastic and thus recover at least a portion of its original size and shape after the removal of the elongating force. Alternatively, wrapsheet 78 can be extensible and can be incorporated in an article including an elastomeric liner 42 and/or outercover 40. In such a configuration, the absorbent structure 70 can be capable of elongating with the outercover 40 and liner 42, and upon removal of the elongating force, the absorbent structure 70 can recover at least a portion of its original size and shape by virtue of the elastomeric outercover 40 and/or liner 42 returning to at least a portion of its original size and shape.

In particular, the absorbent structure 70 can suitably be stretched, laterally and/or longitudinally, by at least 30 percent (to at least 130 percent of an initial (unstretched) width and/or length of the absorbent structure 70). More suitably, the absorbent structure 70 can be stretched laterally and/or longitudinally, by at least 50 percent (to at least 150 percent of the unstretched width or length of the absorbent structure 70). Even more suitably, the absorbent structure 70 can be stretched, laterally and/or longitudinally, by at least 100 percent (to at least 200 percent of the unstretched width or length of the absorbent structure 70). Tension force in the absorbent structure 70 at 50 percent extension is suitably between 50 and 1000 grams, more suitably between 100 and 600 grams, as measured on a 3 inch (7.62 cm) wide piece of the absorbent structure 70.

It is contemplated that additional components or layers may be disposed between the liner 42 and the outercover 40 along with the absorbent structure 70. For example, a surge management layer (not shown) may be located adjacent the absorbent structure 70 (e.g., between the absorbent structure and the liner 42) and attached to various components of the pants 20 such as the absorbent structure 70 and/or the liner 42 by methods known in the art, such as by adhesive, ultrasonic or thermal bonding. A surge management layer helps to decelerate and diffuse surges or gushes of liquid that may be rapidly introduced into the absorbent structure 70 of the article 20. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are incorporated by reference herein.

The pants 20 of the various aspects of the present invention provide a stretchable absorbent article featuring a distinctive absorbent structure 70. The absorbent structure 70 can advantageously accommodate the elongation of the outercover 40 and/or liner 42, while maintaining a desired level of absorbent performance. In particular, the absorbent structure can maintain improved pad integrity under such conditions while suitably containing the absorbent material of the absorbent body 72. Moreover, in certain aspects, the absorbent structure 70 of the present invention can provide a stretchable absorbent while utilizing conventional absorbent materials such as cellulosic fluff and superabsorbent materials.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. An absorbent article comprising:
A stretchable, liquid impermeable outercover;
A stretchable, liquid permeable liner generally superposed with said outercover; and
An absorbent structure disposed between said liner and said outercover, said absorbent structure comprising:
An absorbent body comprising at least two absorbent segments; and
A stretchable, liquid permeable absorbent body wrapsheet substantially surrounding said absorbent body wherein said absorbent body wrapsheet is attached to said absorbent body.

2. The absorbent article of claim 1 wherein said absorbent body defines an absorbent body inner surface and an absorbent body outer surface and wherein said absorbent body wrapsheet is attached to said absorbent body on at least said absorbent body inner surface.

3. The absorbent article of claim 2 wherein said absorbent body wrapsheet is attached to said absorbent body on said absorbent body inner surface and said absorbent body outer surface.

4. The absorbent article of claim 1 wherein said absorbent structure comprises at least 4 absorbent segments.

5. The absorbent article of claim 1 wherein said absorbent article defines a lateral direction and a longitudinal direction perpendicular to said lateral direction, said absorbent structure comprises an absorbent segment matrix, said absorbent segment matrix comprising at least 2 absorbent segments in said lateral direction and at least 5 absorbent segments in said longitudinal direction.

6. The absorbent article of claim 1 wherein said absorbent article defines a lateral direction, a longitudinal direction perpendicular to said lateral direction, and an article plane defined by said lateral direction and said longitudinal direction wherein said absorbent segments are adjacent each other in said article plane.

7. The absorbent article of claim 1 wherein said absorbent body wrapsheet is attached directly to said absorbent body.

8. The absorbent article of claim 1 wherein said absorbent body wrapsheet is attached to said absorbent body with adhesives.

9. The absorbent article of claim 1 wherein said absorbent body wrapsheet is attached to said absorbent body with ultrasonic bonds.

10. The absorbent article of claim 1 wherein said absorbent body wrapsheet is: attached to said absorbent body with pressure bonds.

11. The absorbent article of claim 1 wherein said absorbent body wrapsheet is attached to at least 20 percent of said absorbent segments of said absorbent body.

12. The absorbent article of claim 11 wherein said absorbent body wrapsheet is attached to at least 50 percent of said segments of said absorbent body.

13. The absorbent article of claim 11 wherein said absorbent segments define an absorbent segment area and wherein said absorbent body wrapsheet is attached to said absorbent segments at discrete attachment regions and wherein said discrete attachment regions define an attachment region area that is less than 20 percent of said absorbent segment area.

14. The absorbent article of claim 1 wherein said absorbent body wrapsheet is a single piece of material folded about said absorbent body.

15. The absorbent article of claim 1 wherein said absorbent body wrapsheet comprises multiple pieces of material sandwiching said absorbent body.

16. The absorbent article of claim 1 wherein said absorbent body further comprises a boundary region between each of said absorbent segments and wherein said absorbent segments are spaced in at least one of said lateral direction and said longitudinal direction to provide said boundary region.

17. The absorbent article of claim 1 wherein said absorbent body further comprises a boundary region between said absorbent segments and wherein said absorbent segments define an absorbent segment density and said boundary region defines a boundary region density that is less than said absorbent segment density.

18. The absorbent article of claim 1 wherein said absorbent body further comprises a boundary region between said absorbent segments and wherein said absorbent segments define an absorbent segment basis weight and said boundary region defines a boundary region basis weight that is less than said absorbent segment basis weight.

19. The absorbent article of claim 1 wherein said outercover and said liner are elastomeric.

20. The absorbent article of claim 1 wherein said absorbent body wrapsheet is elastomeric.

21. The absorbent article of claim 1 wherein said absorbent body wrapsheet is treated with a surfactant.

22. An absorbent article comprising:
An elastic, liquid impermeable outercover;
An elastic, liquid permeable liner generally superposed with said outercover; and
An absorbent structure disposed between said liner and said outercover, said absorbent structure comprising:
An absorbent body comprising at least two absorbent segments; and
A stretchable, liquid permeable absorbent body wrapsheet substantially surrounding said absorbent body wherein said absorbent body wrapsheet is attached to said absorbent body.

23. An absorbent article defining a lateral direction and a longitudinal direction, said absorbent article comprising:
An elastic, liquid impermeable outercover;
An elastic, liquid permeable liner generally superposed with said outercover; and
An absorbent structure disposed between said liner and said outercover, said absorbent structure comprising:
An absorbent body comprising an absorbent segment matrix of at least 2 absorbent segments in said lateral direction and at least 5 absorbent segments in said longitudinal direction; and
A stretchable, liquid permeable absorbent body wrapsheet substantially surrounding said absorbent body wherein said absorbent body wrapsheet is attached to said absorbent body.

24. An absorbent article defining a lateral direction and a longitudinal direction, said absorbent article comprising:
An elastic, liquid impermeable outercover;
An elastic, liquid permeable liner generally superposed with said outercover; and
An elastic absorbent structure disposed between said liner and said outercover, said absorbent structure comprising an absorbent body comprising at least two absorbent segments.

25. The absorbent article of claim 24 wherein the absorbent structure is elastic and is capable of elongating in the lateral direction and the longitudinal direction.
